(19) United States
(12) Patent Application Publication
(10) Pub. No.: US 2017/0339949 A1
(43) Pub. Date: Nov. 30, 2017

(54) SUGAR SURFACTANTS AND USE THEREOF IN AGROCHEMICAL COMPOSITIONS

(71) Applicant: Clariant International Ltd., Muttenz (CH)

(72) Inventors: Peter KLUG, Grossostheim (DE); Franz-Xaver SCHERL, Burgkirchen (DE); George Ítalo Pitombeira NUNES, Rheinfelden (CH); Peter BAUR, Schondorf (DE); Roland ARNOLD, Elbtal (DE)

(73) Assignee: Clariant International Ltd., Muttenz (CH)

(21) Appl. No.: 15/534,675

(22) PCT Filed: Dec. 10, 2015
(86) PCT No.: PCT/EP2015/079295

§ 371 Date: Jun. 9, 2017

(30) Foreign Application Priority Data
Dec. 12, 2014 (DE) 10 2014 018 274.6

(51) Int. Cl. A01N 25/30; C07H 15/12; A01N 41/10

(52) U.S. Cl. CPC A01N 25/30; A01N 41/10; C07H 15/12

(57) ABSTRACT

A sugar surfactant of formula (I), where R¹ is H or an alkyl group comprising 1 to 3 carbon atoms and R² is a polyhydroxyhydrocarbyl group comprising a straight hydrocarbyl chain to which at least three hydroxyl groups are directly bonded, preferably a 2,3,4,5,6-pentahydroxyhex-1-yl group, is suitable especially for use in agrochemical adjuvant and active substance compositions.

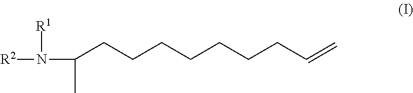

The invention relates to sugar surfactants, to an aqueous adjuvant composition comprising the sugar surfactants, to the use thereof in and for production of agrochemical compositions, and to aqueous, especially agrochemical compositions comprising the sugar surfactants of the invention.

Pesticides (particularly fungicides, herbicides and insecticides) are chemical substances, prepared synthetically or of natural origin, which penetrate into plant cells or tissue or into parasitic organisms in or on the plant and damage and/or destroy them. The majority of pesticides are herbicides. Pesticides are typically used in the form of liquid or solid concentrated preparations (formulations) in agriculture. These are easier for the user to handle or ensure higher efficacy of the active ingredient. The formulations are typically diluted with water prior to use and then deployed by spray application.

Water-soluble concentrates (soluble liquids, abbreviated to SL) are a particularly important form of pesticide preparations. They play a major role particularly in the case of herbicides, pesticides often being used in the form of water-soluble salts which are converted to their alkali metal or ammonium salts by neutralization of the acid form of the herbicides with suitable bases.

A particularly important part is played by the water-soluble salts of herbicides, for example of glyphosate, of glufosinate, or of the auxin herbicides such as 2,4-D or dicamba. They are used preferably as the alkali metal salt or in the form of various ammonium salts, or as a mixture of these salts, usually as aqueous formulations.

It is a general problem in the application of pesticides that only a fraction of the active ingredient displays the desired activity. The greater portion is often lost unutilized, in that the active ingredient does not reach the leaves or roots of the plant on deployment of the spray liquor, but seeps away unutilized in the soil, is washed away by rain or is simply not properly absorbed by the plant. This environmental and economic disadvantage can be reduced by addition of auxiliaries, identified in the context of the present application as “adjuvants”, to pesticide formulations. These auxiliaries are able, for example, to reduce spray drift, improve the wetting of the plant or ensure that the active ingredient sticks on the plant surface for longer or is better absorbed. Particularly in the case of water-soluble pesticides, such as for glyphosate, for example, the nature and also the amount of the adjuvants used have a decisive influence on the activity of the formulation.

By far the most commonly used adjuvants in aqueous herbicide formulations are fatty amine ethoxylates, primarily tallow fatty amine ethoxylates. On account of their toxic and ecotoxicological properties, however, such as the severe eye irritation or the toxicity toward aquatic organisms, these products are classed as objectionable and are increasingly being replaced by adjuvants having a better toxicological and ecotoxicological profile.

Adjuvants which are used in aqueous pesticide formulations are typically in liquid form, i.e. in the form of water-miscible solutions, in order to simplify the production of the pesticide formulation. The adjuvant solutions may comprise water and/or water-miscible solvents which, together with the pesticide, give rise to a homogeneous and storage-stable aqueous formulation. If possible, water is used as solvent, since it is preferable both for reasons of cost and from an environmental standpoint. If necessary, cosolvents capable of improving the solubility or stability are added.

The use of sugar-based surfactants, such as alkyl-N-methylglucosamides, in cleaning products and cosmetic products, for example, is described in the literature (F. W. Lichtenthaler, “Carbohydrates as Organic Raw Materials”, in Ullmann's Encyclopedia of Industrial Chemistry, Wiley-VCH Verlag, 2010).

WO 96/16540 describes pesticide compositions comprising long-chain alkylamides which bear a polyhydroxycarbonyl substituent having at least three hydroxyl groups on the amide nitrogen.

WO 2014/067663 describes aqueous adjuvant compositions which comprise one or more alkylglucamides having a linear or branched C₅-C₉-alkyl group and are suitable for enhancing the action of pesticides and for production of aqueous pesticide compositions.

The demands of adjuvants in aqueous pesticide compositions have grown steadily over the years. As well as high biological efficacy and safety, both from the user's point of view and from an environmental standpoint, more advantageous performance properties are increasingly being demanded. The adjuvants are to enable maximum loading of the formulation with the active ingredient and are to have maximum compatibility with various active ingredients. The formulations have to be storage-stable and have minimum viscosity, in order to assure easier handling, and the maximum emptying of the container. Moreover, good miscibility and rapid dissolution capacity, also and particularly in cold water, in the mixing of the spray liquor is required.

The problem addressed was thus that of providing novel aqueous adjuvant compositions that are highly effective, feature a very advantageous toxicological and ecological profile and have advantageous properties from a performance point of view.

It has been found that sugar surfactants containing a 9-decenoyl radical are particularly suitable for use as adjuvants in agrochemical compositions.

The Invention Therefore Provides a Sugar Surfactant of the Formula (I)

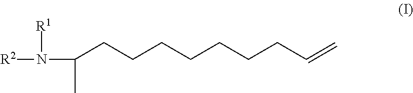

in which
R¹ is H or an alkyl group having 1 to 3 carbon atoms and
R² is a polyhydroxyhydrocarbyl group having a linear, preferably C₄-C₆, hydrocarbyl chain, where at least three hydroxyl groups are bonded directly to the chain, preferably a 2,3,4,5,6-pentahydroxyhex-1-yl group.

The invention further provides a preferably monophasic adjuvant composition comprising

A1) one or more inventive sugar surfactants of the formula (I),
A2) water,
A3) optionally a cosolvent and
A4) optionally one or more ammonium salts
A5) optionally one or more softeners.

The invention further provides for the use of sugar surfactants of the invention and of the composition of the invention as adjuvants for active agrochemical ingredient compositions.

The invention further provides an active ingredient composition, especially active agrochemical ingredient composition, comprising the sugar surfactant of the invention, especially the adjuvant composition of the invention.

The invention likewise provides a method of controlling harmful organisms, wherein the harmful organism or its habitat is brought into contact with an active agrochemical ingredient composition comprising at least one pesticide as active agrochemical ingredient and the adjuvant composition of the invention.

The sugar surfactant of the invention and the adjuvant composition of the invention especially increase the efficacy of electrolyte active ingredients, i.e. pesticides and plant nutrients in the form of water-soluble salts.

Sugar Surfactant

The R² group is preferably obtained from a reducing sugar by reductive amination. Suitable reducing sugars are, for example, glucose, fructose, maltose, lactose, galactose, mannose and xylose. Suitable starting materials, as well as the sugars mentioned, are also high dextrose corn syrup, high fructose corn syrup and high maltose corn syrup. These corn syrup types especially give rise to mixtures of various sugar components for the sugar surfactants of the formula (I).

In the sugar surfactants of the formula (I), R¹ is preferably a C₁-C₃-alkyl group, more preferably methyl.

R² is preferably —CH₂—(CHOH)ₙ—CH₂OH,
—CH₂—(CH₂OH)(CHOH)ₙ₋₁—CH₂OH, —CH₂—(CHOH)₂(CHOR³)(CHOH)—CH₂OH,
where

n is 3, 4 or 5, preferably 4, and

R³ is H or a cyclic mono- or polysaccharide.

R² is more preferably —CH₂—(CHOH)₄—CH₂OH.

A most preferred sugar surfactant (I) is N-methyl-N-dec-9-enoylglucamine (Ia)

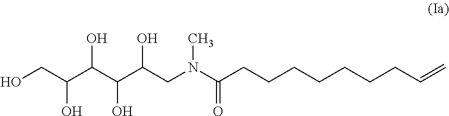 [SMILES: C=CCCCCCCCC(=O)N(C)CC(O)C(O)C(O)C(O)CO]

The sugar residue R² here is most preferably derived from D-glucose.

The sugar surfactants (I) of the invention are prepared, for example, by reaction of the corresponding sugar amines obtainable from reductive sugars, such as N-methylglucamine, with the likewise commercially available methyl 9-decenoate. A corresponding preparation method is described, for example, in EP-A 0 550 637.

The invention therefore also provides a process for preparing sugar surfactants of the formula (I), wherein a sugar amine of the formula (II)

R¹R²NH (II)

where R¹ and R² have the definitions given in formula (I)
is reacted with methyl 9-decenoate, preferably in a protic solvent such as 1,2-propylene glycol.

Adjuvant Composition

Adjuvant compositions of the invention include, as well as at least one sugar surfactant (I) (A1) and water (A2), optionally a cosolvent (A3) and optionally one or more ammonium salts (A4) and optionally one or more softeners (A5).

The proportion of the inventive sugar surfactants (I) (component (A1)) in the adjuvant composition is preferably 5% to 90% by weight, more preferably 10% to 80% by weight and especially preferably 10% to 30% by weight.

As component (A2), the adjuvant compositions comprise water. Useful water includes, for example, demineralized water, groundwater, seawater or tap water. The water preferably has a hardness below 15° dH (German hardness).

The water content is preferably 20% to 89% by weight, more preferably 25% to 70% by weight and especially preferably 30% to 60% by weight.

The cosolvent (A3) optionally present may either be present as a secondary component from the preparation process for the alkylglucamide or have been added subsequently to the adjuvant composition. The cosolvent may be a single solvent or a mixture of two or more solvents. Suitable solvents for this purpose are all polar solvents that are compatible with the aqueous composition and form a homogeneous phase. Suitable cosolvents are, for example, monohydric alcohols such as methanol, ethanol, propanols, butanols, benzyl alcohol or polyhydric alcohols such as ethylene glycol, diethylene glycol, propylene glycol or glycerol, or polyglycols such as polyethylene glycols, polypropylene glycols or mixed polyalkylene glycols (PAGs). Further suitable solvents are ethers, for example propylene glycol mono- or dimethyl ether, dipropylene glycol mono- or dimethyl ether, amides, for example N-methyl- or N-ethylpyrrolidone, N,N-dimethyllactamide, -caprylamide or -decanamide.

Particularly suitable cosolvents are mono- or polyhydric alcohols, and especially suitable cosolvents are di- or trihydric alcohols such as propylene glycol, glycerol or polyethylene glycols, polypropylene glycols or mixed polyalkylene glycols (PAGs).

Most preferably, the adjuvant compositions comprise, as component (A3), propylene glycol or dipropylene glycol, and combinations of the two with one another or with polypropylene glycol or polyethylene glycol having up to 10 repeat units. Propylene glycol is especially preferred.

The propylene glycol content—if present—is preferably 1% to 30% by weight, more preferably 2% to 10% by weight and especially preferably 2% to 5% by weight.

The proportion of the cosolvent in the composition—if present—is typically 10 to 250 g/L, preferably 20 to 200 g/L and more preferably 30 to 150 g/L.

The cosolvent can additionally contribute to stabilization of the adjuvant compositions by virtue of it, for example, increasing the cold or heat stability or having a positive effect on further performance properties such as viscosity.

As component (A4), the adjuvant compositions optionally comprise one or more and preferably one or two water-soluble ammonium salts, more preferably one water-soluble ammonium salt.

Preferred ammonium salts are ammonium sulfate, ammonium nitrate, ammonium nitrate urea, ammonium phosphate, ammonium citrate, ammonium thiosulfate and/or ammonium chloride, more preferably ammonium sulfate, ammonium nitrate, ammonium citrate and/or ammonium nitrate urea; very particular preference is given to ammonium sulfate.

If component (A4) is present, the adjuvant compositions contain preferably 5% to 60% by weight, more preferably 10% to 50% by weight and especially preferably 20% to 50% by weight of the one or more ammonium salts.

As component (A5), the adjuvant compositions of the invention optionally comprise a softener. “Softener” is understood in accordance with the invention to mean a substance that eliminates the alkaline earth metal cations dissolved in water, such as Ca²⁺ and Mg²⁺, or masks them by complex formation.

Softeners used with preference are alkali metal phosphates, alkali metal citrates and alkali metal sulfates such as Na₂SO₄, glycerol sulfates, phosphonates, nitrilotriacetate, aminopolycarboxylic acids such as EDTA and DTPA, aminopolyphosphonic acids such as EDTMP and HEDP, gluconates, monocarbamide dihydrogensulfate, polycarboxylates such as polyacrylates, and polyphosphonates.

In a preferred embodiment, the adjuvant composition of the invention comprises one or more softeners.

In a further embodiment, the adjuvant composition of the invention does not comprise any softener.

In a further embodiment, the adjuvant compositions of the invention may comprise one or more further auxiliaries (A6), which may, for example, be preservatives, surfactants, defoamers, functional polymers or additional adjuvants. Examples of auxiliaries can be found further down.

In a preferred embodiment, the adjuvant compositions consist of components (A1) to (A6), more preferably (A1) to (A5).

The aqueous adjuvant compositions of the sugar surfactants of the formula (I) are suitable as adjuvants especially in aqueous active agrochemical ingredient formulations for improving the biological activity of pesticides such as herbicides, insecticides, fungicides, acaricides, bactericides, molluscicides, nematicides and rodenticides, and also plant nutrients and plant fortifiers.

The invention therefore also provides for the use of the aqueous adjuvant compositions for enhancing the biological activity of active agrochemical ingredients, especially of herbicides.

The aqueous adjuvant compositions are outstandingly suitable for production of storage-stable active agrochemical ingredient compositions having advantageous properties.

The invention therefore also provides for the use of the compositions of the invention for production of aqueous active agrochemical ingredient compositions. Standard methods of producing such compositions are known to those skilled in the art.

The invention further provides aqueous active ingredient compositions comprising
(W1) one or more inventive sugar surfactants (I),
(W2) water,
(W3) one or more water-soluble active ingredients, especially active agrochemical ingredients,
(W4) optionally a cosolvent
(W5) optionally one or more ammonium salts,
(W6) optionally one or more softeners,
(W7) optionally one or more auxiliaries
(W8) optionally one or more water-insoluble active ingredients, especially pesticides.

In respect of component (W1), sugar surfactant, the statements made with regard to component (A1) of the adjuvant composition are analogously applicable.

In respect of component (W2), water, the statements made with regard to component (A2) of the adjuvant composition are analogously applicable.

Useful active ingredients (W3), aside from the preferred active agrochemical ingredients, are, for example, active pharmaceutical and cosmetic ingredients.

Water-soluble active agrochemical ingredients (W3) in the context of the invention are understood to mean those active agrochemical ingredients which have a solubility at room temperature (25° C.) of more 50 g/L and preferably more than 100 g/L in water. Particular preference is given to active agrochemical ingredients which have a solubility of at least 90% by weight at 20° C. at the use concentration.

Preferred active agrochemical ingredients include pesticides, plant nutrients and plant stimulants, more preferably herbicides, insecticides, acaricides, nematicides, repellents, fungicides, bactericides, plant growth regulators, plant nutrients and plant fortifiers.

The compositions of the invention are especially suitable for combinations comprising one or more of the following water-soluble active ingredients:

acifluorfen, aminopyralid, amitrole, asulam, benazolin, bentazon, bialaphos, bispyribac, bromacil, bromoxynil, bicyclopyron, chloramben, clopyralid, 2,4-D, 2,4-DB, dicamba, dichlorprop, difenzoquat, diquat, endothal, fenoxaprop, flamprop, florasulam, flumiclorac, fluoroglycofen, fluroxypyr, fomesafen, fosamine, glufosinate, glyphosate, imazameth, imazamethabenz, imazamox, imazapic, imazapyr, imazaquin, imazethapyr, MCPA, MCPB, mecoprop, mesotrione, nicosulfuron, octanoic acid, pelargonic acid, picloram, quizalofop, 2,3,6-TBA, sulcotrione, tembotrione and triclopyr, more preferably 2,4-D, bentazon, dicamba, fomesafen, glyphosate, glufosinate, MCPA, mesotrione, paraquat and sulcotrione, preferably from the water-soluble salts of glyphosate and dicamba.

Preferred salts here are, for example, acifluorfen-sodium, bialafos-sodium, bispyridac-sodium, glufosinate-ammonium, glufosinate-P-ammonium, glufosinate-P-sodium, glyphosate-isopropylammonium, glyphosate-trimesium, imazamox-ammonium, imazapyr-isopropylammonium, imazaquinammonium, imazethapyr-ammonium, MCPB-sodium, mecocrop-sodium, mecocrop-P-dimethylammonium and mecocrop-P-potassium.

Preferred water-soluble active agrochemical ingredients are also plant nutrients, especially inorganic cationic nutrients such as iron, magnesium and potassium, and inorganic cationic micronutrients such as boron, copper, manganese, molybdenum and zinc, where nutrients and micronutrients may be in the form of salts and/or chelate compounds.

Preference is likewise given to plant growth regulators, for example natural plant hormones, especially abscisic acid, jasmonic acid, salicylic acid and esters thereof, kinetin and brassinosteroids.

Further preferred plant stimulants include substances that act as plant strengtheners in order to reduce the effect of stress factors such as heat, cold, drought, salt, oxygen deficiency or waterlogging on plant growth. Examples include glycine betaine (betaine), choline, phosphate salts such as potassium phosphate, and silicates.

The compositions of the invention may additionally, in the formulation or else the spray liquor, comprise further water-soluble active ingredients (W8) that may be in dispersed form, for example.

There follow further examples of active agrochemical ingredients that may be present in dissolved form as component (W3) or may be combination partners for these pesticides (component (W8)).

Active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoendesaturase, photosystem I, photosystem II, protoporphyrinogen oxidase usable, as described, for example, in Weed Research 26 (1986) 441 445 or “The Pesticide Manual”, 16th edition, The British Crop Protection Council and the Royal Soc. of Chemistry, 2012 and literature cited therein. Examples of known herbicides or plant growth regulators which can be combined with the inventive compounds include the active ingredients which follow (the compounds are designated by the “common name” according to the International Organization for Standardization (ISO) or by the chemical name or by the code number) and always encompass all use forms, such as acids, salts, esters and isomers, such as stereoisomers and optical isomers. One administration form or else, in some cases, more than one administration form is mentioned by way of example:

acetochlor, acibenzolar, acibenzolar-S-methyl, aclonifen, alachlor, allidochlor, alloxydim, alloxydim-sodium, ametryn, amicarbazone, amidochlor, amidosulfuron, aminocyclopyrachlor, aminocyclopyrachlor-potassium, aminocyclopyrachlor-methyl, ammonium sulfamate, ancymidol, anilofos, atrazine, aviglycin, azafenidin, azimsulfuron, aziprotryn, beflubutamid, benazolinethyl, bencarbazone, benfluralin, benfuresate, bensulide, bensulfuron, bensulfuron-methyl, benzfendizone, benzobicyclon, benzofenap, benzofluor, benzoylprop, benzyladenin, bifenox, bilanafos, bilanafos-sodium, bromobutide, bromofenoxim, bromuron, buminafos, busoxinone, butachlor, butafenacil, butamifos, butenachlor, butralin, butroxydim, butylate, cafenstrole, carbaryl, carbetamide, carfentrazone, carfentrazone-ethyl, carvone, chlorocholine chloride, chlomethoxyfen, chlorazifop, chlorazifop-butyl, chlorbromuron, chlorbufam, chlorfenac, chlorfenac-sodium, chlorfenprop, chlorflurenol, chlorflurenol-methyl, chloridazon, chlorimuron, chlorimuron-ethyl, chlormequat chloride, chlornitrofen, 4-chlorophenoxyacetic acid, chlorophthalim, chlorpropham, chlorthal-dimethyl, chlorotoluron, chlorsulfuron, cinidon, cinidonethyl, cinmethylin, cinosulfuron, clethodim, clodinafop, clodinafop-propargyl, clofencet, clomazone, clomeprop, cloprop, cloransulam, cloransulam-methyl, cloxyfonac, cumyluron, cyanamide, cyanazine, cyclanilide, cycloate, cyclosulfamuron, cycloxydim, cycluron, cyhalofop, cyhalofop-butyl, cyperquat, cyprazine, cyprazole, cytokinine, daimuron/dymron, dalapon, daminozide, dazomet, n-decanol, desmedipham, desmetryn, detosyl pyrazolate (DTP), diallate, diaminozid, dichlobenil, dichlorprop-P, diclofop, diclofop-methyl, diclofop-P-methyl, diclosulam, diethatyl, diethatyl-ethyl, difenoxuron, diflufenican, diflufenzopyr, diflufenzopyr-sodium, dikegulac-sodium, dimefuron, dimepiperate, dimethachlor, dimethametryn, dimethenamid, dimethenamid-P, dimethipin, dimetrasulfuron, dinitramine, dinoseb, dinoterb, diphenamid, diisopropylnaphthalene, dipropetryn, diquat dibromide, dithiopyr, diuron, DNOC, eglinazine-ethyl, EPTC, esprocarb, ethalfluralin, ethametsulfuron, ethametsulfuron-methyl, ethylnaphthyl acetate, ethephon, ethidimuron, ethiozin, ethofumesate, ethoxyfen, ethoxyfen-ethyl, ethoxysulfuron, etobenzanid, F-5331, i.e. N-[2-chloro-4-fluoro-5-[4-(3-fluoropropyl)-4,5-dihydro-5-oxo-1H-tetrazol-1-yl]phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoprop, fenoxaprop-ethyl, fenoxaprop-P-ethyl, fenoxasulfone, fentrazamide, fenuron, flamprop, flamprop-M-isopropyl, flamprop-M-methyl, flazasulfuron, fluazifop, fluazifop-P, fluazifop-butyl, fluazifop-P-butyl, fluazolate, flucarbazone, flucarbazone-sodium, flucetosulfuron, fluchloralin, flufenacet (thiafluamide), flufenpyr, flufenpyrethyl, flumetralin, flumetsulam, flumiclorac-pentyl, flumioxazin, flumipropyn, fluometuron, fluorodifen, fluoroglycofen-ethyl, flupoxam, flupropacil, flupropanate, flupyrsulfuron, flupyrsulfuron-methyl-sodium, flurenol, flurenol-butyl, fluridone, flurochloridone, fluroxypyr-meptyl, flurprimidol, flurtamone, fluthiacet, fluthiacet-methyl, fluthiamide, foramsulfuron, forchlorfenuron, furyloxyfen, gibberellic acid, H-9201, i.e. O-(2,4-dimethyl-6-nitrophenyl)-O-ethylisopropylphosphorus amidothioate, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz-methyl, imazosulfuron, inabenfide, indanofan, indaziflam, indoleacetic acid (IAA), 4-indol-3-ylbutyric acid (IBA), iodosulfuron, iodosulfuronmethyl-sodium, iofensulfuron, iofensulfuron-sodium, ioxynil, ipfencarbazone, isocarbamid, isopropalin, isoproturon, isouron, isoxaben, isoxachlortole, isoxaflutole, isoxapyrifop, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, karbutilate, ketospiradox, lactofen, lenacil, linuron, maleic hydrazide, MCPB-methyl, -ethyl, mecoprop-butotyl, mecoprop-P-butotyl, mecoprop-P-2-ethylhexyl, mefenacet, mefluidide, mepiquat chloride, mesosulfuron, mesosulfuron-methyl, methabenzthiazuron, metam, metamifop, metamitron, metazachlor, metazasulfuron, methazole, methiopyrsulfuron, methiozolin, methoxyphenone, methyldymron, 1-methylcyclopropene, methyl isothiocyanate, metobenzuron, metobromuron, metolachlor, S-metolachlor, metosulam, metoxuron, metribuzin, metsulfuron, metsulfuron-methyl, molinate, monalide, monocarbamide, monocarbamide dihydrogensulfate, monolinuron, monosulfuron, monosulfuron ester, monuron, MT-128, i.e. 6-chloro-N-[(2E)-3-chloroprop-2-en-1-yl]-5-methyl-N-phenylpyridazin-3-amine, MT-5950, i.e. N-[3-chloro-4-(1-methylethyl)phenyl]-2-methylpentanamide, NGGC-011, 1-naphthylacetic acid (NAA), naphthylacetamide (NAAm), 2-naphthoxyacetic acid, naproanilide, napropamide, naptalam, NC-310, i.e. 4-(2,4-dichlorobenzoyl)-1-methyl-5-benzyloxypyrazole, neburon, nipyraclofen, nitralin, nitrofen, nitroguaiacolate, nitrophenolate-sodium (isomer mixture), nitrofluorfen, nonanoic acid, norflurazon, orbencarb, orthosulfamuron, oryzalin, oxadiargyl, oxadiazon, oxasulfuron, oxaziclomefone, oxyfluorfen, paclobutrazol, paraquat, paraquat dichloride, pendimethalin, pendralin, penoxsulam, pentanochlor, pentoxazone, perfluidone, pethoxamid, phenisopham, phenmedipham, phenmedipham-ethyl, picolinafen, pinoxaden, piperophos, pirifenop, pirifenop-butyl, pretilachlor, primisulfuron, primisulfuron-methyl, probenazole, profluazol, procyazine, prodiamine, prifluraline, profoxydim, prohexadione, prohexadione-calcium, prohydrojasmone, prometon, prometryn, propachlor, propanil, propaquizafop, propazine, propham, propisochlor, propoxycarbazone, propoxycarbazone-sodium, propyrisulfuron, propyzamide, prosulfalin, prosulfocarb, prosulfuron, prynachlor, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofopethyl, quizalofop-P, quizalofop-P-ethyl, quizalofop-P-tefuryl, rimsulfuron, saflufenacil, secbumeton, sethoxydim, siduron, simazine, simetryn, SN-106279, i.e. methyl (2R)-2-({7-[2-chloro-4-(trifluoromethyl)phenoxy]-2-naphthyl}oxy)propanoate, sulfallate (CDEC), sulfentrazone, sulfometuron, sulfometuron-methyl, sulfo-sulfuron, SW-065, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, tebutam, tebuthiuron, tecnazene, tefuryltrione, tepraloxydim, terbacil, terbucarb, terbuchlor, terbumeton, terbuthylazine, terbutryn, thenylchlor, thiafluamide, thiazafluron, thiazopyr, thidiazimin, thidiazuron, thiencarbazone, thiencarbazone-methyl, thifensulfuron, thifensulfuron-methyl, thiobencarb, tiocarbazil, topramezone, tralkoxydim, triafamone, triallate, triasulfuron, triaziflam, triazofenamide, tribenuron, tribenuron-methyl, tribufos, trichloroacetic acid (TCA), tridiphane, trietazine, trifloxysulfuron, trifloxysulfuron-sodium, trifluralin, triflusulfuron, triflusulfuron-methyl, trimeturon, trinexapac, trinexapac-ethyl, tritosulfuron, tsitodef, uniconazole, uniconazole-P, vernolate, ZJ-0862, i.e. 3,4-dichloro-N-{2-[(4,6-dimethoxypyrimidin-2-yl)oxy]benzyl}aniline, and the following compounds:

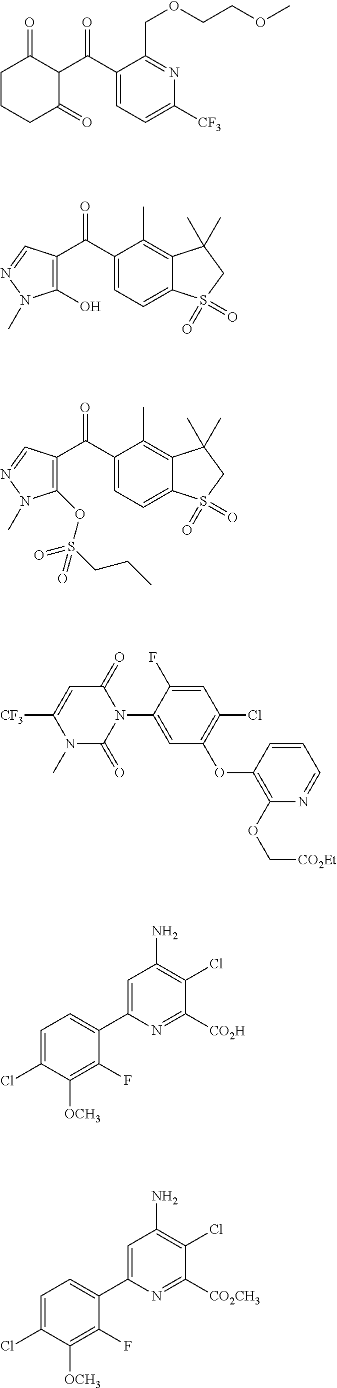 [SMILES: COCCOCc1nc(C(F)(F)F)ccc1C(=O)C1C(=O)CCCC1=O ; Cc1c(C(=O)c2cnn(C)c2O)ccc2c1C(C)(C)CS2(=O)=O ; CCCS(=O)(=O)Oc1c(C(=O)c2ccc3c(c2C)C(C)(C)CS3(=O)=O)cnn1C ; CCOC(=O)COc1ncccc1Oc1cc(-n2c(=O)cc(C(F)(F)F)n(C)c2=O)c(F)cc1Cl ; COc1c(Cl)ccc(-c2cc(N)c(Cl)c(C(=O)O)n2)c1F ; COC(=O)c1nc(-c2ccc(Cl)c(OC)c2F)cc(N)c1Cl]

Further examples of plant nutrients include customary inorganic or organic fertilizers for supplying plants with macro- and/or micronutrients.

Examples of fungicides include:

(1) Ergosterol biosynthesis inhibitors, for example aldimorph, azaconazole, bitertanol, bromuconazole, cyproconazole, diclobutrazole, difenoconazole, diniconazole, diniconazole-M, dodemorph, dodemorph acetate, epoxiconazole, etaconazole, fenarimol, fenbuconazole, fenhexamid, fenpropidin, fenpropimorph, fluquinconazole, flurprimidol, flusilazole, flutriafol, furconazole, furconazole-cis, hexaconazole, imazalil, imazalil sulfate, imibenconazole, ipconazole, metconazole, myclobutanil, naftifin, nuarimol, oxpoconazole, paclobutrazole, pefurazoate, penconazole, piperalin, prochloraz, propiconazole, prothioconazole, pyributicarb, pyrifenox, quinconazole, simeconazole, spiroxamine, tebuconazole, terbinafine, tetraconazole, triadimefon, triadimenol, tridemorph, triflumizole, triforine, triticonazole, uniconazole, uniconazole-p, viniconazole, voriconazole, 1-(4-chlorophenyl)-2-(1H-1,2,4-triazol-1-yl)cycloheptanol, methyl 1-(2,2-dimethyl-2,3-dihydro-1H-inden-1-yl)-1H-imidazole-5-carboxylate, N′-{5-(difluoromethyl)-2-methyl-4-[3-(trimethylsilyl)propoxy]phenyl}-N-ethyl-N-methylimidoformamide, N-ethyl-N-methyl-N′-{2-methyl-5-(trifluoromethyl)-4-[3-(trimethylsilyl)propoxy]phenyl}imidoformamide and O-[1-(4-methoxyphenoxy)-3,3-dimethylbutan-2-yl]1H-imidazole-1-carbothioate.
(2) Respiration inhibitors (respiratory chain inhibitors), for example bixafen, boscalid, carboxin, diflumetorim, fenfuram, fluopyram, flutolanil, fluxapyroxad, furametpyr, furmecyclox, isopyrazam mixture of the syn-epimeric racemate 1RS,4SR,9RS and of the anti-epimeric racemate 1RS,4SR,9SR, isopyrazam (anti-epimeric racemate), isopyrazam (anti-epimeric enantiomer 1R,4S,9S), isopyrazam (anti-epimeric enantiomer 1S,4R,9R), isopyrazam (syn-epimeric racemate 1RS,4SR,9RS), isopyrazam (syn-epimeric enantiomer 1R,4S,9R), isopyrazam (syn-epimeric enantiomer 1S,4R,9S), mepronil, oxycarboxin, penflufen, penthiopyrad, sedaxane, thifluzamid, 1-methyl-N-[2-(1,1,2,2-tetrafluoroethoxy)phenyl]-3-(trifluoromethyl)-1H-pyrazole-4-carboxamide, 3-(difluoromethyl)-1-methyl-N-[2-(1,1,2,2-tetrafluoroethoxy)phenyl]-1H-pyrazole-4-carboxamide, 3-(difluoromethyl)-N-[4-fluoro-2-(1,1,2,3,3,3-hexafluoropropoxy)phenyl]-1-methyl-1H-pyrazole-4-carboxamide, N-[1-(2,4-dichlorophenyl)-1-methoxypropan-2-yl]-3-(difluoromethyl)-1-methyl-1H-pyrazole-4-carboxamide, 5,8-difluoro-N-[2-(2-fluoro-4-{[4-(trifluoromethyl)pyridin-2-yl]oxy}phenyl)ethyl]quinazolin-4-amine, N-[9-(dichloromethylene)-1,2,3,4-tetrahydro-1,4-methanonaphthalen-5-yl]-3-(difluoromethyl)-1-methyl-1H-pyrazole-4-carboxamide, N-[(1S,4R)-9-(dichloromethylene)-1,2,3,4-tetrahydro-1,4-methanonaphthalen-5-yl]-3-(difluoromethyl)-1-methyl-1H-pyrazole-4-carboxamide and N-[(1R,4S)-9-(dichloromethylene)-1,2,3,4-tetrahydro-1,4-methanonaphthalen-5-yl]-3-(difluoromethyl)-1-methyl-1H-pyrazole-4-carboxamide.
(3) Respiration inhibitors (respiratory chain inhibitors) acting on complex III of the respiratory chain, for example ametoctradin, amisulbrom, azoxystrobin, cyazofamid, coumethoxystrobin, coumoxystrobin, dimoxystrobin, enestroburin, famoxadone, fenamidone, fenoxystrobin, fluoxastrobin, kresoxim-methyl, metominostrobin, orysastrobin, picoxystrobin, pyraclostrobin, pyrametostrobin, pyraoxystrobin, pyribencarb, triclopyricarb, trifloxystrobin, (2E)-2-(2-{[6-(3-chloro-2-methylphenoxy)-5-fluoropyrimidin-4-yl]oxy}phenyl)-2-(methoxyimino)-N-methylethanamide, (2E)-2-(methoxyimino)-N-methyl-2-(2-{[({(1E)-1-[3-(trifluoromethyl)phenyl]ethylidene}amino)oxy]methyl}phenyl)ethanamide, (2E)-2-(methoxyimino)-N-methyl-2-{2-[(E)-({1-[3-(trifluoromethyl)phenyl]ethoxy}imino)methyl]phenyl}ethanamide, (2E)-2-{2-[({[(1E)-1-(3-{[(E)-1-fluoro-2-phenylethenyl]oxy}phenyl)ethylidene]amino}oxy)methyl]phenyl}-2-(methoxyimino)-N-methylethanamide, (2E)-2-{2-[({[(2E,3E)-4-(2,6-dichlorophenyl)but-3-en-2-ylidene]amino}oxy)methyl]phenyl}-2-(methoxyimino)-N-methylethanamide, 2-chloro-N-(1,1,3-trimethyl-2,3-dihydro-1H-inden-4-yl)pyridine-3-carboxamide, 5-methoxy-2-methyl-4-(2-{[({(1E)-1-[3-(trifluoromethyl)phenyl]ethylidene}amino)oxy]methyl}phenyl)-2,4-dihydro-3H-1,2,4-triazol-3-one, methyl (2E)-2-{2-[({cyclopropyl[(4-methoxyphenyl)imino]methyl}sulfanyl)methyl]phenyl}-3-methoxyprop-2-enoate, N-(3-ethyl-3,5,5-trimethylcyclohexyl)-3-(formylamino)-2-hydroxybenzamide, 2-{2-[(2,5-dimethylphenoxy)methyl]phenyl}-2-methoxy-N-methylacetamide and (2R)-2-{2-[(2,5-dimethylphenoxy)methyl]phenyl}-2-methoxy-N-methylacetamide.
(4) Mitosis and cell division inhibitors, for example benomyl, carbendazim, chlorfenazole, diethofencarb, ethaboxam, fluopicolide, fuberidazole, pencycuron, thiabendazole, thiophanate-methyl, thiophanate, zoxamide, 5-chloro-7-(4-methylpiperidin-1-yl)-6-(2,4,6-trifluorophenyl)[1,2,4]triazolo[1,5-a]pyrimidine and 3-chloro-5-(6-chloropyridin-3-yl)-6-methyl-4-(2,4,6-trifluorophenyl)pyridazine.
(5) Compounds with multisite activity, for example Bordeaux mixture, captafol, captan, chlorothalonil, copper preparations such as copper hydroxide, copper naphthenate, copper oxide, copper oxychloride, copper sulfate, dichlofluanid, dithianon, dodine, dodine free base, ferbam, fluorofolpet, folpet, guazatine, guazatine acetate, iminoctadine, iminoctadine albesilate, iminoctadine triacetate, mancopper, mancozeb, maneb, metiram, metiram zinc, oxine-copper, propamidine, propineb, sulfur and sulfur preparations, for example calcium polysulfide, thiram, tolylfluanid, zineb and ziram.
(6) Resistance inductors, for example acibenzolar-S-methyl, isotianil, probenazole and tiadinil.
(7) Amino acid and protein biosynthesis inhibitors, for example andoprim, blasticidin-S, cyprodinil, kasugamycin, kasugamycin hydrochloride hydrate, mepanipyrim, pyrimethanil and 3-(5-fluoro-3,3,4,4-tetramethyl-3,4-dihydroisoquinolin-1-yl)quinoline.
(8) Inhibitors of ATP production, for example fentin acetate, fentin chloride, fentin hydroxide and silthiofam.
(9) Cell wall synthesis inhibitors, for example benthiavalicarb, dimethomorph, flumorph, iprovalicarb, mandipropamid, polyoxins, polyoxorim, validamycin A and valifenalate.
(10) Lipid and membrane synthesis inhibitors, for example biphenyl, chloroneb, dicloran, edifenphos, etridiazole, iodocarb, iprobenfos, isoprothiolane, propamocarb, propamocarb hydrochloride, prothiocarb, pyrazophos, quintozene, tecnazene and tolclofos-methyl.
(11) Melanin biosynthesis inhibitors, for example carpropamid, diclocymet, fenoxanil, fthalide, pyroquilon, tricyclazole and 2,2,2-trifluoroethyl {3-methyl-1-[(4-methylbenzoyl)amino]butan-2-yl}carbamate.
(12) Nucleic acid synthesis inhibitors, for example benalaxyl, benalaxyl-M (kiralaxyl), bupirimate, clozylacon, dimethirimol, ethirimol, furalaxyl, hymexazol, metalaxyl, metalaxyl-M (mefenoxam), ofurace, oxadixyl and oxolinic acid.
(13) Signal transduction inhibitors, for example chlozolinate, fenpiclonil, fludioxonil, iprodione, procymidone, quinoxyfen and vinclozolin.
(14) Decouplers, for example binapacryl, dinocap, ferimzone, fluazinam and meptyldinocap.
(15) Further compounds, for example benthiazole, bethoxazin, capsimycin, carvone, chinomethionat, pyriofenone (chlazafenone), cufraneb, cyflufenamid, cymoxanil, cyprosulfamide, dazomet, debacarb, dichlorophen, diclomezine, difenzoquat, difenzoquat methylsulfate, diphenylamine, ecomat, fenpyrazamine, flumetover, fluoromide, flusulfamide, flutianil, fosetyl-aluminum, fosetyl-calcium, fosetyl-sodium, hexachlorobenzene, irumamycin, methasulfocarb, methyl isothiocyanate, metrafenon, mildiomycin, natamycin, nickel dimethyldithiocarbamate, nitrothal-isopropyl, octhilinone, oxamocarb, oxyfenthiin, pentachlorophenol and salts thereof, phenothrin, phosphoric acid and salts thereof, propamocarb-fosetylate, propanosine-sodium, proquinazid, pyrimorph, (2E)-3-(4-tert-butylphenyl)-3-(2-chloropyridin-4-yl)-1-(morpholin-4-yl)prop-2-en-1-one, (2Z)-3-(4-tert-butylphenyl)-3-(2-chloropyridin-4-yl)-1-(morpholin-4-yl)prop-2-en-1-one, pyrrolnitrin, tebufloquin, tecloftalam, tolnifanid, triazoxide, trichlamide, zarilamide, (3S,6S,7R,8R)-8-benzyl-3-[({3-[(isobutyryloxy)methoxy]-4-methoxypyridin-2-yl}carbonyl)amino]-6-methyl-4,9-dioxo-1,5-dioxonan-7-yl 2-methylpropanoate, 1-(4-{4-[(5R)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[(5S)-5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-{4-[5-(2,6-difluorophenyl)-4,5-dihydro-1,2-oxazol-3-yl]-1,3-thiazol-2-yl}piperidin-1-yl)-2-[5-methyl-3-(trifluoromethyl)-1H-pyrazol-1-yl]ethanone, 1-(4-methoxyphenoxy)-3,3-dimethylbutan-2-yl 1H-imidazole-1-carboxylate, 2,3,5,6-tetrachloro-4-(methylsulfonyl)pyridine, 2,3-dibutyl-6-chlorothieno[2,3-d]pyrimidin-4(3H)-one, 2,6-dimethyl-1H,5H-[1,4]dithiino[2,3-c:5,6-c′]dipyrrole-1,3,5,7(2H,6H)-tetrone, 2-[5-methyl-3-(trifluoromethyl)-1H-pyrazol-1-yl]-1-(4-{4-[(5R)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-(4-{4-[(5S)-5-phenyl-4,5-dihydro-1,2-oxazol-3-yl]-1,3-thiazol-2-yl}piperidin-1-yl)ethanone, 2-[5-methyl-3-(trifluoromethyl)-1H-pyrazol-1-yl]-1-{4-[4-(5-phenyl-4,5-dihydro-1,2-oxazol-3-yl)-1,3-thiazol-2-yl]piperidin-1-yl}ethanone, 2-butoxy-6-iodo-3-propyl-4H-chromen-4-one, 2-chloro-5-[2-chloro-1-(2,6-difluoro-4-methoxyphenyl)-4-methyl-1H-imidazol-5-yl]pyridine, 2-phenylphenol and salts thereof, 3-(4,4,5-trifluoro-3,3-dimethyl-3,4-dihydroisoquinolin-1-yl)quinoline, 3,4,5-trichloropyridine-2,6-dicarbonitrile, 3-[5-(4-chlorophenyl)-2,3-dimethyl-1,2-oxazolidin-3-yl]pyridine, 3-chloro-5-(4-chlorophenyl)-4-(2,6-difluorophenyl)-6-methylpyridazine, 4-(4-chlorophenyl)-5-(2,6-difluorophenyl)-3,6-dimethylpyridazine, 5-amino-1,3,4-thiadiazole-2-thiol, 5-chloro-N′-phenyl-N′-(prop-2-yn-1-yl)thiophene-2-sulfonohydrazide, 5-fluoro-2-[(4-fluorobenzyl)oxy]pyrimidin-4-amine, 5-fluoro-2-[(4-methylbenzyl)oxy]pyrimidin-4-amine, 5-methyl-6-octyl[1,2,4]triazolo[1,5-a]pyrimidin-7-amine, ethyl (2Z)-3-amino-2-cyano-3-phenylprop-2-enoate, N′-(4-{[3-(4-chlorobenzyl)-1,2,4-thiadiazol-5-yl]oxy}-2,5-dimethylphenyl)-N-ethyl-N-methylimidoformamide, N-(4-chlorobenzyl)-3-[3-methoxy-4-(prop-2-yn-1-yloxy)phenyl]propanamide, N-[(4-chlorophenyl)(cyano)methyl]-3-[3-methoxy-4-(prop-2-yn-1-yloxy)phenyl]propanamide, N-[(5-bromo-3-chloropyridin-2-yl)methyl]-2,4-dichloropyridine-3-carboxamide, N-[1-(5-bromo-3-chloropyridin-2-yl)ethyl]-2,4-dichloropyridine-3-carboxamide, N-[1-(5-bromo-3-chloropyridin-2-yl)ethyl]-2-fluoro-4-iodopyridine-3-carboxamide, N-{(E)-[(cyclopropylmethoxy)imino][6-(difluoromethoxy)-2,3-difluorophenyl]methyl}-2-phenylacetamide, N—{(Z)-[(cyclopropylmethoxy)imino][6-(difluoromethoxy)-2,3-difluorophenyl]methyl}-2-phenylacetamide, N′-{4-[(3-tert-butyl-4-cyano-1,2-thiazol-5-yl)oxy]-2-chloro-5-methylphenyl}-N-ethyl-N-methylimidoformamide, N-methyl-2-(1-{[5-methyl-3-(trifluoromethyl)-1H-pyrazol-1-yl]acetyl}piperidin-4-yl)-N-(1,2,3,4-tetrahydronaphthalen-1-yl)-1,3-thiazole-4-carboxamide, N-methyl-2-(1-{[5-methyl-3-(trifluoromethyl)-1H-pyrazol-1-yl]acetyl}piperidin-4-yl)-N-[(1R)-1,2,3,4-tetrahydronaphthalen-1-yl]-1,3-thiazole-4-carboxamide, N-methyl-2-(1-{[5-methyl-3-(trifluoromethyl)-1H-pyrazol-1-yl]acetyl}piperidin-4-yl)-N-[(1S)-1,2,3,4-tetrahydronaphthalen-1-yl]-1, 3-thiazole-4-carboxamide, pentyl {6-[({[(1-methyl-1H-tetrazol-5-yl)(phenyl)methylidene]amino}oxy)methyl]pyridin-2-yl}carbamate, phenazine-1-carboxylic acid, quinolin-8-ol, quinolin-8-ol sulfate (2:1) and tert-butyl {6-[({[(1-methyl-1H-tetrazol-5-yl)(phenyl)methylene]amino}oxy)methyl]pyridin-2-yl}carbamate.
(16) Further compounds, for example 1-methyl-3-(trifluoromethyl)-N-[2′-(trifluoromethyl)biphenyl-2-yl]-1H-pyrazole-4-carboxamide, N-(4′-chlorobiphenyl-2-yl)-3-(difluoromethyl)-1-methyl-1H-pyrazole-4-carboxamide, N-(2′,4′-dichlorobiphenyl-2-yl)-3-(difluoromethyl)-1-methyl-1H-pyrazole-4-carboxamide, 3-(difluoromethyl)-1-methyl-N-[4′-(trifluoromethyl)biphenyl-2-yl]-1H-pyrazole-4-carboxamide, N-(2′,5′-difluorobiphenyl-2-yl)-1-methyl-3-(trifluoromethyl)-1H-pyrazole-4-carboxamide, 3-(difluoromethyl)-1-methyl-N-[4′-(prop-1-yn-1-yl)biphenyl-2-yl]-1H-pyrazole-4-carboxamide, 5-fluoro-1,3-dimethyl-N-[4′-(prop-1-yn-1-yl)biphenyl-2-yl]-1H-pyrazole-4-carboxamide, 2-chloro-N-[4′-(prop-1-yn-1-yl)biphenyl-2-yl]pyridine-3-carboxamide, 3-(difluoromethyl)-N-[4′-(3,3-dimethylbut-1-yn-1-yl)biphenyl-2-yl]-1-methyl-1H-pyrazole-4-carboxamide, N-[4′-(3,3-dimethylbut-1-yn-1-yl)biphenyl-2-yl]-5-fluoro-1,3-dimethyl-1H-pyrazole-4-carboxamide, 3-(difluoromethyl)-N-(4′-ethynylbiphenyl-2-yl)-1-methyl-1H-pyrazole-4-carboxamide, N-(4′-ethynylbiphenyl-2-yl)-5-fluoro-1,3-dimethyl-1H-pyrazole-4-carboxamide, 2-chloro-N-(4′-ethynylbiphenyl-2-yl)pyridine-3-carboxamide, 2-chloro-N-[4′-(3,3-dimethylbut-1-yn-1-yl)biphenyl-2-yl]pyridine-3-carboxamide, 4-(difluoromethyl)-2-methyl-N-[4′-(trifluoromethyl)biphenyl-2-yl]-1,3-thiazole-5-carboxamide, 5-fluoro-N-[4′-(3-hydroxy-3-methylbut-1-yn-1-yl)biphenyl-2-yl]-1,3-dimethyl-1H-pyrazole-4-carboxamide, 2-chloro-N-[4′-(3-hydroxy-3-methylbut-1-yn-1-yl)biphenyl-2-yl]pyridine-3-carboxamide, 3-(difluoromethyl)-N-[4′-(3-methoxy-3-methylbut-1-yn-1-yl)biphenyl-2-yl]-1-methyl-1H-pyrazole-4-carboxamide, 5-fluoro-N-[4′-(3-methoxy-3-methylbut-1-yn-1-yl)biphenyl-2-yl]-1,3-dimethyl-1H-pyrazole-4-carboxamide, 2-chloro-N-[4′-(3-methoxy-3-methylbut-1-yn-1-yl)biphenyl-2-yl]pyridine-3-carboxamide, (5-bromo-2-methoxy-4-methylpyridin-3-yl)(2,3,4-trimethoxy-6-methylphenyl)methanone, N-[2-(4-{[3-(4-chlorophenyl)prop-2-yn-1-yl]oxy}-3-methoxyphenyl)ethyl]-N2-(methylsulfonyl)valinamide, 4-oxo-4-[(2-phenylethyl)amino]butanoic acid and but-3-yn-1-yl {6-[({[(Z)-(1-methyl-1H-tetrazol-5-yl)(phenyl)methylene]amino}oxy)methyl]pyridin-2-yl}carbamate.

All fungicides (1) to (16) mentioned may, when they are capable on account of their functional groups, optionally form salts with suitable bases or acids.

Examples of bactericides include the following:

bronopol, dichlorophen, nitrapyrin, nickel dimethyldithiocarbamate, kasugamycin, octhilinon, furancarboxylic acid, oxytetracycline, probenazole, streptomycin, tecloftalam, copper sulfate and other copper preparations.

Examples of insecticides, acaricides and nematicides include the following:

(1) Acetylcholinesterase (AChE) inhibitors, such as carbamates, e.g. alanycarb, aldicarb, bendiocarb, benfuracarb, butocarboxim, butoxycarboxim, carbaryl, carbofuran, carbosulfan, ethiofencarb, fenobucarb, formetanate, furathiocarb, isoprocarb, methiocarb, methomyl, metolcarb, oxamyl, pirimicarb, propoxur, thiodicarb, thiofanox, triazamate, trimethacarb, XMC and xylylcarb; or organophosphates, e.g. acephate, azamethiphos, azinphos-ethyl, azinphos-methyl, cadusafos, chlorethoxyfos, chlorfenvinphos, chlormephos, chlorpyrifos, chlorpyrifos-methyl, coumaphos, cyanophos, demeton-S-methyl, diazinon, dichlorvos/DDVP, dicrotophos, dimethoate, dimethylvinphos, disulfoton, EPN, ethion, ethoprophos, famphur, fenamiphos, fenitrothion, fenthion, fosthiazate, heptenophos, imicyafos, isofenphos, isopropyl O-(methoxyaminothiophosphoryl) salicylate, isoxathion, malathion, mecarbam, methamidophos, methidathion, mevinphos, monocrotophos, naled, omethoate, oxydemeton-methyl, parathion, parathion-methyl, phenthoate, phorate, phosalone, phosmet, phosphamidon, phoxim, pirimiphos-methyl, profenofos, propetamphos, prothiofos, pyraclofos, pyridaphenthion, quinalphos, sulfotep, tebupirimfos, temephos, terbufos, tetrachlorvinphos, thiometon, triazophos, triclorfon and vamidothion.
(2) GABA-gated chloride channel antagonists, for example cyclodiene-organochlorines, e.g. chlordane and endosulfan; or phenylpyrazoles (fiproles), e.g. ethiprole and fipronil.
(3) Sodium channel modulators/voltage-gated sodium channel blockers, for example pyrethroids, e.g. acrinathrin, allethrin, d-cis-trans allethrin, d-trans allethrin, bifenthrin, bioallethrin, bioallethrin s-cyclopentenyl isomer, bioresmethrin, cycloprothrin, cyfluthrin, beta-cyfluthrin, cyhalothrin, lambda-cyhalothrin, gamma-cyhalothrin, cypermethrin, alpha-cypermethrin, beta-cypermethrin, theta-cypermethrin, zeta-cypermethrin, cyphenothrin [(1R)-trans isomers], deltamethrin, empenthrin [(EZ)-(1R) isomers], esfenvalerate, etofenprox, fenpropathrin, fenvalerate, flucythrinate, flumethrin, tau-fluvalinate, halfenprox, imiprothrin, kadethrin, permethrin, phenothrin [(1R)-trans isomer], prallethrin, pyrethrins (pyrethrum), resmethrin, silafluofen, tefluthrin, tetramethrin, tetramethrin [(1R) isomers)], tralomethrin and transfluthrin; or DDT; or methoxychlor.
(4) Nicotinergic acetylcholine receptor (nAChR) agonists, for example neonicotinoids, e.g. acetamiprid, clothianidin, dinotefuran, imidacloprid, nitenpyram, thiacloprid and thiamethoxam; or nicotine.
(5) Allosteric activators of the nicotinergic acetylcholine receptor (nAChR), for example spinosyns, e.g. spinetoram and spinosad.
(6) Chloride channel activators, for example avermectins/milbemycins, e.g. abamectin, emamectin benzoate, lepimectin and milbemectin.
(7) Juvenile hormone imitators, for example juvenile hormone analogs e.g. hydroprene, kinoprene and methoprene; or fenoxycarb; or pyriproxyfen.
(8) Active ingredients with unknown or nonspecific mechanisms of action, for example alkyl halides, e.g. methyl bromide and other alkyl halides; or chloropicrin; or sulfuryl fluoride; or borax; or tartar emetic.
(9) Selective antifeedants, for example pymetrozine; or flonicamid.
(10) Mite growth inhibitors, for example clofentezine, hexythiazox and diflovidazin; or etoxazole.
(11) Microbial disruptors of the insect gut membrane, for example Bacillus thuringiensis subspecies israelensis, Bacillus sphaericus, Bacillus thuringiensis subspecies aizawai, Bacillus thuringiensis subspecies kurstaki, Bacillus thuringiensis subspecies tenebrionis, and BT plant proteins: Cry1Ab, Cry1Ac, Cry1 Fa, Cry2Ab, mCry3A, Cry3Ab, Cry3Bb, Cry34/35Ab1.
(12) Oxidative phosphorylation inhibitors, ATP disruptors, for example diafenthiuron; or organotin compounds, e.g. azocyclotin, cyhexatin and fenbutatin oxide; or propargite; or tetradifon.
(13) Oxidative phosphorylation decouplers that interrupt the H proton gradient, for example chlorfenapyr, DNOC and sulfluramid.
(14) Nicotinergic acetylcholine receptor antagonists, for example bensultap, cartap hydrochloride, thiocyclam, and thiosultap-sodium.
(15) Chitin biosynthesis inhibitors, type 0, for example bistrifluron, chlorfluazuron, diflubenzuron, flucycloxuron, flufenoxuron, hexaflumuron, lufenuron, novaluron, noviflumuron, teflubenzuron and triflumuron.
(16) Chitin biosynthesis inhibitors, type 1, for example buprofezin.
(17) Molting disruptors, dipteran, for example cyromazine.
(18) Ecdysone receptor agonists, for example chromafenozide, halofenozide, methoxyfenozide and tebufenozide.
(19) Octopaminergic agonists, for example amitraz.
(20) Complex-III electron transport inhibitors, for example hydramethylnon; or acequinocyl; or fluacrypyrim.
(21) Complex-I electron transport inhibitors, for example METI acaricides, e.g. fenazaquin, fenpyroximate, pyrimidifen, pyridaben, tebufenpyrad and tolfenpyrad; or rotenone (Derris).
(22) Voltage-dependent sodium channel blockers, for example indoxacarb; or metaflumizone.
(23) Inhibitors of acetyl-CoA carboxylase, for example tetronic and tetramic acid derivatives, e.g. spirodiclofen, spiromesifen and spirotetramat.
(24) Complex-IV electron transport inhibitors, for example phosphines, e.g. aluminum phosphide, calcium phosphide, phosphine and zinc phosphide; or cyanide.
(25) Complex-II electron transport inhibitors, for example cyenopyrafen.
(26) Ryanodine receptor effectors, for example diamides, e.g. chlorantraniliprole and flubendiamide.

Further active ingredients with an unknown mechanism of action, for example amidoflumet, azadirachtin, benclothiaz, benzoximate, bifenazate, bromopropylate, chinomethionat, cryolite, cyantraniliprole (Cyazypyr), cyflumetofen, dicofol, diflovidazin, fluensulfone, flufenerim, flufiprole, fluopyram, fufenozide, imidaclothiz, iprodione, pyridalyl, pyrifluquinazon and iodomethane; and additionally preparations based on Bacillus firmus (I-1582, BioNeem, Votivo) and the following known active compounds:

3-bromo-N-{2-bromo-4-chloro-6-[(1-cyclopropylethyl)carbamoyl]phenyl}-1-(3-chloropyridin-2-yl)-1H-pyrazole-5-carboxamide (known from WO 2005/077934), 4-{[(6-bromopyrid-3-yl)methyl](2-fluoroethyl)amino}furan-2(5H)-one (known from WO 2007/115644), 4-{[(6-fluoropyrid-3-yl)methyl](2,2-difluoroethyl)amino}furan-2(5H)-one (known from WO 2007/115644), 4-{[(2-chloro-1,3-thiazol-5-yl)methyl](2-fluoroethyl)amino}furan-2(5H)-one (known from WO 2007/115644), 4-{[(6-chloropyrid-3-yl)methyl](2-fluoroethyl)amino}furan-2(5H)-one (known from WO 2007/115644), 4-{[(6-chloropyrid-3-yl)methyl](2,2-difluoroethyl)amino}furan-2(5H)-one (known from WO 2007/115644), 4-{[(6-chloro-5-fluoropyrid-3-yl)methyl](methyl)amino}furan-2(5H)-one (known from WO 2007/115643), 4-{[(5,6-dichloropyrid-3-yl)methyl](2-fluoroethyl)amino}furan-2(5H)-one (known from WO 2007/115646), 4-{[(6-chloro-5-fluoropyrid-3-yl)methyl](cyclopropyl)amino}furan-2(5H)-one (known from WO 2007/115643), 4-{[(6-chloropyrid-3-yl)methyl](cyclopropyl)amino}furan-2(5H)-one (known from EP-A 0 539 588), 4-{[(6-chloropyrid-3-yl)methyl](methyl)amino}furan-2(5H)-one (known from EP-A 0 539 588), {[1-(6-chloropyridin-3-yl)ethyl](methyl)oxido-λ4-sulfanylidene}cyanamide (known from WO 2007/149134) and its diastereomers {[(1R)-1-(6-chloropyridin-3-yl)ethyl](methyl)oxido-λ4-sulfanylidene}cyanamide (A) and {[(1S)-1-(6-chloropyridin-3-yl)ethyl](methyl)oxido-λ4-sulfanylidene}cyanamide (B) (likewise known from WO 2007/149134) and also sulfoxaflor (likewise known from WO 2007/149134) and its diastereomers [(R)-methyl(oxido){(1R)-1-[6-(trifluoromethyl)pyridin-3-yl]ethyl}-λ4-sulfanylidene]cyanamide (A1) and [(S)-methyl(oxido){(1S)-1-[6-(trifluoromethyl)pyridin-3-yl]ethyl}-λ4-sulfanylidene]cyanamide (A2), identified as diastereomer group A (known from WO 2010/074747, WO 2010/074751), [(R)-methyl(oxido){(1S)-1-[6-(trifluoromethyl)pyridin-3-yl]ethyl}-λ4-sulfanylidene]cyanamide (B1) and [(S)-methyl(oxido){(1R)-1-[6-(trifluoromethyl)pyridin-3-yl]ethyl}-λ4-sulfanylidene]cyanamide (B2), identified as diastereomer group B (likewise known from WO 2010/074747, WO 2010/074751) and 11-(4-chloro-2,6-dimethylphenyl)-12-hydroxy-1,4-dioxa-9-azadispiro[4.2.4.2]tetradec-11-en-10-one (known from WO 2006/089633), 3-(4′-fluoro-2,4-dimethylbiphenyl-3-yl)-4-hydroxy-8-oxa-1-azaspiro[4.5]dec-3-en-2-one (known from WO 2008/067911), 1-{2-fluoro-4-methyl-5-[(2,2,2-trifluoroethyl)sulfinyl]phenyl}-3-(trifluoromethyl)-1H-1,2,4-triazol-5-amine (known from WO 2006/043635), [(3S,4aR,12R,12aS,12bS)-3-[(cyclopropylcarbonyl)oxy]-6,12-dihydroxy-4,12b-dimethyl-11-oxo-9-(pyridin-3-yl)-1,3,4,4a,5,6,6a,12,12a,12b-decahydro-2H,11H-benzo[f]pyrano[4,3-b]chromen-4-yl]methyl cyclopropanecarboxylate (known from WO 2008/066153), 2-cyano-3-(difluoromethoxy)-N,N-dimethylbenzenesulfonamide (known from WO 2006/056433), 2-cyano-3-(difluoromethoxy)-N-methylbenzenesulfonamide (known from WO 2006/100288), 2-cyano-3-(difluoromethoxy)-N-ethylbenzenesulfonamide (known from WO 2005/035486), 4-(difluoromethoxy)-N-ethyl-N-methyl-1,2-benzothiazol-3-amine 1,1-dioxide (known from WO 2007/057407), N-[1-(2,3-dimethylphenyl)-2-(3,5-dimethylphenyl)ethyl]-4,5-dihydro-1,3-thiazol-2-amine (known from WO 2008/104503), {1′-[(2E)-3-(4-chlorophenyl)prop-2-en-1-yl]-5-fluorospiro[indole-3,4′-piperidin]-1(2H)-yl}(2-chloropyridin-4-yl)methanone (known from WO 2003/106457), 3-(2,5-dimethylphenyl)-4-hydroxy-8-methoxy-1,8-diazaspiro[4.5]dec-3-en-2-one (known from WO 2009/049851), 3-(2,5-dimethylphenyl)-8-methoxy-2-oxo-1,8-diazaspiro[4.5]dec-3-en-4-yl ethyl carbonate (known from WO 2009/049851), 4-(but-2-yn-1-yloxy)-6-(3,5-dimethylpiperidin-1-yl)-5-fluoropyrimidine (known from WO 2004/099160), (2,2,3,3,4,4,5,5-octafluoropentyl)(3,3,3-trifluoropropyl)malononitrile (known from WO 2005/063094), (2,2,3,3,4,4,5,5-octafluoropentyl)(3,3,4,4,4-pentafluorobutyl)malononitrile (known from WO 2005/063094), 8-[2-(cyclopropylmethoxy)-4-(trifluoromethyl)phenoxy]-3-[6-(trifluoromethyl)pyridazin-3-yl]-3-azabicyclo[3.2.1]octane (known from WO 2007/040280), 2-ethyl-7-methoxy-3-methyl-6-[(2,2,3,3-tetrafluoro-2,3-dihydro-1,4-benzodioxin-6-yl)oxy]quinolin-4-yl methyl carbonate (known from JP2008/110953), 2-ethyl-7-methoxy-3-methyl-6-[(2,2,3,3-tetrafluoro-2,3-dihydro-1,4-benzodioxin-6-yl)oxy]quinolin-4-yl acetate (known from JP2008/110953), PF1364 (CAS Reg. No. 1204776-60-2) (known from JP 2010/018586), 5-[5-(3,5-dichlorophenyl)-5-(trifluoromethyl)-4,5-dihydro-1,2-oxazol-3-yl]-2-(1H-1,2,4-triazol-1-yl)benzonitrile (known from WO 2007/075459), 5-[5-(2-chloropyridin-4-yl)-5-(trifluoromethyl)-4,5-dihydro-1,2-oxazol-3-yl]-2-(1H-1,2,4-triazol-1-yl)benzonitrile (known from WO 2007/075459), 4-[5-(3,5-dichlorophenyl)-5-(trifluoromethyl)-4,5-dihydro-1,2-oxazol-3-yl]-2-methyl-N-{2-oxo-2-[(2,2,2-trifluoroethyl)amino]ethyl}benzamide (known from WO 2005/085216), 4-{[(6-chloropyridin-3-yl)methyl](cyclopropyl)amino}-1,3-oxazol-2(5H)-one, 4-{[(6-chloropyridin-3-yl)methyl](2,2-difluoroethyl)amino}-1,3-oxazol-2(5H)-one, 4-{[(6-chloropyridin-3-yl)methyl](ethyl)amino}-1,3-oxazol-2(5H)-one, 4-{[(6-chloropyridin-3-yl)methyl](methyl)amino}-1,3-oxazol-2(5H)-one (all known from WO 2010/005692), NNI-0711 (known from WO2002/096882), 1-acetyl-N-[4-(1,1,1,3,3,3-hexafluoro-2-methoxypropan-2-yl)-3-isobutylphenyl]-N-isobutyryl-3,5-dimethyl-1H-pyrazole-4-carboxamide (known from WO 2002/096882), methyl 2-[2-({[3-bromo-1-(3-chloropyridin-2-yl)-1H-pyrazol-5-yl]carbonyl}amino)-5-chloro-3-methyl benzoyl]-2-methylhydrazinecarboxylate (known from WO 2005/085216), methyl 2-[2-({[3-bromo-1-(3-chloropyridin-2-yl)-1H-pyrazol-5-yl]carbonyl}amino)-5-cyano-3-methylbenzoyl]-2-ethylhydrazinecarboxylate (known from WO 2005/085216), methyl 2-[2-({[3-bromo-1-(3-chloropyridin-2-yl)-1H-pyrazol-5-yl]carbonyl}amino)-5-cyano-3-methylbenzoyl]-2-methylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-1,2-diethylhydrazinecarboxylate (known from WO 2005/085216), methyl 2-[3,5-dibromo-2-({[3-bromo-1-(3-chloropyridin-2-yl)-1H-pyrazol-5-yl]carbonyl}amino)benzoyl]-2-ethylhydrazinecarboxylate (known from WO 2005/085216), (5RS,7RS; 5RS,7SR)-1-(6-chloro-3-pyridylmethyl)-1,2,3,5,6,7-hexahydro-7-methyl-8-nitro-5-propoxyimidazo[1,2-a]pyridine (known from WO 2007/101369), 2-{6-[2-(5-fluoropyridin-3-yl)-1,3-thiazol-5-yl]pyridin-2-yl}pyrimidine (known from WO 2010/006713), 2-{6-[2-(pyridin-3-yl)-1,3-thiazol-5-yl]pyridin-2-yl}pyrimidine (known from WO 2010/006713), 1-(3-chloropyridin-2-yl)-N-[4-cyano-2-methyl-6-(methylcarbamoyl)phenyl]-3-{[5-(trifluoromethyl)-1H-tetrazol-1-yl]methyl}-1H-pyrazole-5-carboxamide (known from WO 2010/069502), 1-(3-chloropyridin-2-yl)-N-[4-cyano-2-methyl-6-(methylcarbamoyl)phenyl]-3-{[5-(trifluoromethyl)-2H-tetrazol-2-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-1H-tetrazol-1-yl]methyl}-1H-pyrazole-5-carboxamide (known from WO 2010/069502), N-[2-(tert-butylcarbamoyl)-4-cyano-6-methylphenyl]-1-(3-chloropyridin-2-yl)-3-{[5-(trifluoromethyl)-2H-tetrazol-2-yl]methyl}-1H-pyrazole-5-carboxamide (known from WO 2010/069502), and (1E)-N-[(6-chloropyridin-3-yl)methyl]-N′-cyano-N-(2,2-difluoroethyl)ethanimidamide (known from WO 2008/009360).

The active ingredients referred to here by their common names are known and are described, for example, in the Pesticide Manual (16th ed., British Crop Protection Council 2012) or can be searched for on the Internet (e.g. http://www.alanwood.net/pesticides).

The active agrochemical ingredients of component (W3) and/or (W8) may also be a combination of two or more active agrochemical ingredients. Such combinations are of significance especially when the aim is, for example, to broaden the spectrum of action of the active agrochemical ingredient composition or to better suppress resistances to particular active agrochemical ingredients.

The combination of two or more active agrochemical ingredients in one formulation is a difficult undertaking. The active ingredients are typically incompatible with one another and the aqueous mixtures are therefore not phase-stable. However, the adjuvant compositions of the invention are of good suitability for stabilization of such basically incompatible compositions.

In a further embodiment of the invention, therefore, the active agrochemical ingredient compositions of the invention comprise at least two water-soluble active agrochemical ingredients of component (W3).

The at least two water-soluble active agrochemical ingredients are preferably herbicides selected from glyphosate, glufosinate, 2,4-D, dicamba and fomesafen.

Particularly preferred compositions are those where the water-soluble active agrochemical ingredients of component (W3) comprise the combinations of the two herbicides glyphosate and 2,4-D, glyphosate and dicamba, glyphosate and fomesafen, glyfosate and glufosinate, 2,4-D and dicamba, glufosinate and 2,4-D, and glufosinate and dicamba.

The aim in the formulation of aqueous active agrochemical ingredient compositions is to load the composition with a maximum concentration of active ingredient. This reduces packaging, transport, storage and disposal costs. Therefore, an adjuvant composition should be capable of enabling stable high-load active agrochemical ingredient compositions, called “high-load formulations”. This is possible in a surprisingly efficient manner with the alkylglucamides of the formula (I).

In a preferred embodiment of the invention, the amount of the one or more water-soluble active agrochemical ingredients of component (W3) in the compositions of the invention is more than 100 g/L, preferably more than 200 g/L and more preferably more than 300 g/L. These figures are based on the total weight of the active agrochemical ingredient composition of the invention and, in the case of active agrochemical ingredients which are used in the form of their water-soluble salts (such as typically glyphosate or 2,4-D, for example), on the amount of free acid, called the acid equivalent (a.e.).

In a further preferred embodiment of the invention, the amount of the one or more sugar surfactants of the formula (I) in the active agrochemical ingredient compositions of the invention is 20 to 250 g/L, preferably 40 to 200 g/L and more preferably 50 to 150 g/L. These figures are based on the total amount of the active agrochemical ingredient composition of the invention.

Typically, the sugar surfactants of the formula (I) are used in the form of solutions. For clarification, it should be mentioned here that the figures given above are based on the active content of the sugar surfactants of the formula (I) in the solution.

A particularly important criterion for the storage stability of aqueous active agrochemical ingredient compositions, such as glyphosate and 2,4-D formulations, is phase stability. A composition is regarded as having sufficient phase stability when it remains homogeneous over a wide temperature range and when there is no formation of two or more separate phases or precipitates (formation of a further solid phase). Phase stability is the crucial prerequisite for a storage-stable formulation both at elevated temperature, as can occur, for example, in the case of storage in the sun or in warm countries, and at low temperature, for example in winter or in cold climatic regions.

It is a feature of the active agrochemical ingredient compositions of the invention that they are phase-stable even at a temperature of preferably greater than 55° C., more preferably of greater than 70° C. and especially preferably of greater than 80° C.

It is also a feature of the active agrochemical ingredient compositions of the invention that they are phase-stable even at a temperature of preferably less than 10° C., more preferably of less than 0° C. and especially preferably of less than −10° C.

The pH of the active agrochemical ingredient compositions is typically within the range from 3.5 to 8.0, preferably 4.0 to 7.0 and more preferably 4.5 to 6.5 (measured as a 1% by weight aqueous dilution). The pH is determined primarily by the pH values of the solutions of the aqueous pesticides, which take the form of salts of weak acids. By adding acids or bases, it is possible to adjust the pH to another value different than the original pH of the mixture.

The high salt stability of the active agrochemical ingredient composition of the invention in an aqueous medium even at high active ingredient and salt concentration is a great performance advantage. It also enables incorporation of agrochemical salts, for example fertilizers, into the composition.

The active ingredient compositions of the invention optionally comprise one or more cosolvents (component (W4)).

In a preferred embodiment, the active ingredient compositions comprise at least one cosolvent. In a further embodiment, the active ingredient compositions do not comprise any cosolvent.

In respect of component (W4), the statements made with regard to component (A3) of the adjuvant composition are analogously applicable.

The active ingredient compositions of the invention optionally comprise one or more ammonium salts (component (W5)).

In a preferred embodiment, the active ingredient compositions comprise at least one ammonium salt. In a further embodiment, the active ingredient compositions do not comprise any ammonium salt.

In respect of component (W5), the statements made with regard to component (A4) of the adjuvant composition are analogously applicable.

The active ingredient compositions of the invention optionally comprise one or more softeners (component (W5)).

In a preferred embodiment, the active ingredient compositions comprise at least one softener. In a further embodiment, the active ingredient compositions do not comprise any softener.

In respect of component (W6), the statements made with regard to component (A5) of the adjuvant composition are analogously applicable.

The active agrochemical ingredient compositions of the invention optionally comprise one or more further auxiliaries (W7), where these may, for example, be preservatives, further surfactants other than the sugar surfactant (I), defoamers, functional polymers or additional adjuvants.

Preservatives used may be organic acids and esters thereof, for example ascorbic acid, ascorbyl palmitate, sorbate, benzoic acid, methyl and propyl 4-hydroxybenzoate, propionates, phenol, for example 2-phenylphenate, 1,2-benzisothiazolin-3-one, formaldehyde, sulfurous acid and salts thereof.

The surfactants may generally be any nonionic, amphoteric, cationic or anionic surfactants that are compatible with the composition.

Examples of nonionic surfactants are ethoxylates and alkoxylates of relatively long-chain aliphatic or aromatic alcohols, fatty amine ethoxylates, relatively long-chain etheramine alkoxylates, (optionally ethoxylated) sorbitan esters, alkyl polyglycosides. Suitable amphoteric surfactants include long-chain alkyl dimethylbetaines or alkyldimethylamine oxides, or alkyldimethylamine amidopropylamine oxides. Among the anionic surfactants, for example, ether sulfates of ethoxylated fatty alcohols, reaction products of (optionally ethoxylated) long-chain alcohols with phosphoric acid derivatives are suitable. “Long-chain” is understood to mean linear or branched hydrocarbon chains having at least 6 and at most 22 carbon atoms.

In a preferred embodiment, the active ingredient composition does not comprise any further sugar surfactants aside from the sugar surfactant of the formula (I).

Suitable defoamers are fatty acid alkyl ester alkoxylates, organopolysiloxanes such as polydimethylsiloxanes and mixtures thereof with microfine, optionally silanized silica; perfluoroalkylphosphonates and -phosphinates, paraffins, waxes and microcrystalline waxes, and mixtures thereof with silanized silica. Also advantageous are mixtures of various foam inhibitors, for example those of silicone oil, paraffin oil and/or waxes.

The functional polymers which may be present in the pesticide composition of the invention are high molecular weight compounds of synthetic or natural origin having a molar mass of greater than 10 000. The functional polymers may act, for example, as anti-drift agents or increase rain resistance.

In a further preferred embodiment of the invention, the active agrochemical ingredient compositions of the invention comprise, as component (W7), one or more further adjuvants as usable in a known manner in aqueous pesticide compositions.

These are preferably fatty amine ethoxylates, etheramine ethoxylates, alkyl betaines or amidoalkyl betaines, amine oxides or amidoalkylamine oxides, alkyl polyglycosides or copolymers of glycerol, coconut fatty acid and phthalic acid.

These adjuvants are known from the literature as adjuvants in aqueous pesticide compositions and are described, for example, in WO2009/029561.

In a further preferred embodiment of the invention, the active agrochemical ingredient compositions of the invention are in the form of concentrate formulations which are diluted prior to use, especially with water (for example “ready-to-use”, “in-can” or “built-in” formulations), and contain the one or more water-soluble active agrochemical ingredients of component (W3) generally in amounts of 5% to 80% by weight, preferably of 10% to 70% by weight and more preferably of 20% to 60% by weight, the one or more sugar surfactants of the formula (I) (W1) in amounts of 1% to 25% by weight, preferably of 2% to 20% by weight and more preferably of 3% to 15% by weight, the ammonium salt (W5)—if present—in amounts of 5% to 50% by weight, preferably 10% to 40% by weight and more preferably 10% to 35% by weight, cosolvent (W4) in amounts of 1% to 30% by weight, preferably of 2% to 10% by weight and more preferably of 2% to 5% by weight and—if present—softeners (W6) in amounts of 1% to 50% by weight, preferably 2% to 25% by weight and more preferably 3% to 10% by weight. These figures are based on the overall concentrate formulation and, in the case of pesticides which are used in the form of their water-soluble salts, on the amount of free acid, called the acid equivalent (a.e.).

The pesticide compositions of the invention are preferably deployed to the fields in the form of spray liquors. The spray liquors are produced by diluting concentrate formulations with a defined amount of water.

In a further preferred embodiment of the invention, the active agrochemical ingredient compositions of the invention are in the form of spray liquors and contain 0.001% to 10% by weight, preferably 0.02% to 3% by weight and more preferably 0.025% to 2% by weight of the one or more water-soluble active agrochemical ingredients of component (W3) and 0.001% to 3% by weight, preferably 0.005% to 1% by weight and more preferably 0.01% to 0.5% by weight of the one or more sugar surfactants of the formula (I) (W1). The figures given are based on the overall spray liquor and, in the case of active agrochemical ingredients which are used in the form of their water-soluble salts, on the amount of free acid, called the acid equivalent (a.e.).

The invention further relates to the use of the active agrochemical ingredient compositions of the invention where the active ingredient is a pesticide for control and/or for abatement of weeds, fungal diseases or insect infestation. Preference is given to the use of such pesticide compositions for control and/or for abatement of weeds.

These uses can preferably also take place in what is known as the tankmix method. In this case, the one or more water-soluble active agrochemical ingredients of component (W3) and the one or more sugar surfactants of the formula (I) and the water may thus also take the form of a “tankmix” preparation. In such a preparation, both the one or more water-soluble active agrochemical ingredients and the one or more sugar surfactants of the formula (I), the latter optionally together with further adjuvants, are present separately from one another. The two preparations are mixed with one another prior to deployment, generally shortly beforehand, giving rise to an active agrochemical ingredient composition of the invention.

### WORKING EXAMPLES

The invention is further illustrated to the person skilled in the art hereinafter by examples, but these should in no way be considered as a restriction to the embodiments detailed.

### Example 1: Production of an Aqueous Sugar Surfactant Composition of the Invention

The solution comprising 50% active N-methyl-N-dec-9-enoylglucamine substance was prepared as follows: first of all, 9-decenoic acid (Aldrich) was reacted with an excess of methanol to give methyl 9-decenoate. The methyl 9-decenoate was distilled and then reacted with N-methylglucamine in the presence of 1,2-propylene glycol as solvent according to EP 0 550 637 and obtained as a solid consisting of 90% active substance and 10% 1,2-propylene glycol. This solid was dissolved at 40 to 50° C. in water, so as to give a solution with a 50% content of N-methyl-N-dec-9-enoylglucamine. This is a clear colorless solution.

### Example 2

Increasing the absorption of systemic active ingredients using the example of mesotrione and test system for measurement of the promotion of penetration of active ingredients

Surfactants can promote the absorption of (active) ingredients through membranes such as skin, films or the plant cuticle. As a “finite-dose” application, it is known for the single administration or application of a solution, cream, gel etc. to a membrane that the absorption of active ingredient can be influenced by some additives such as surfactants even after wetting. This effect is independent of the interfacial effect in water, is often highly concentration-dependent and takes place for the most part after evaporation of water and any solvents present rather than as a result of the interaction, for example, with active ingredient, membrane and environmental factors. For various surfactants, it is observed after addition to active ingredient preparations that the penetration of a particular active ingredient is promoted to an enormous degree by some surfactants, whereas others are entirely ineffective (Cronfeld, P, Lader, K. Baur, P. (2001). Classification of Adjuvants and Adjuvant Blends by Effects on Cuticular Penetration, Pesticide Formulations and Application Systems: Twentieth Volume, ASTM STP 1400, A. K. Viets, R. S. Tann, J. C. Mueninghoff, eds., American Society for Testing and Materials, West Conshohocken, Pa. 2001).

The potential of the test substances, which is independent of the surfactant action, to promote foliar absorption of active agrochemical ingredients was determined in membrane penetration tests with apple leaf cuticles for mesotrione. Mesotrione is the most important modern active herbicide ingredient and, like other important herbicides (for instance 2,4-D or dicamba), is a weak organic acid having a pKa of 3.1 in the range of 2-6 and has good systemic characteristics after absorption via the leaf cuticle. With a number of such acids or electrolyte active ingredients, consistently excellent promotion of absorption has already been found with Synergen GA (N-methyl-N-octanoyl/decanoylglucamine), especially together with ammonium sulfate in the case of use of water with alkaline earth metal contents, such as calcium ion contents, that are potentially antagonistic to acidic herbicides. The plant cuticle is a lipophilic solubility membrane (lipid membrane) without pores or holes, and the results described are also expected for other nonporous lipophilic solubility membranes with these or other electrolyte active ingredients. The principle of the method has been published (e.g. WO-A-2005/194844; Baur, 1997; Baur, Grayson and Schönherr 1999; Baur, Bodelon and Lowe, 2012), and only the specifics and differences in the method are elucidated hereinafter. The leaf cuticles were enzymatically isolated in the manner described in the literature from apple leaves of orchard trees in a commercial fruit growing facility near Frankfurt am Main in 2011. The stomata-free cuticles were first dried under air and then installed into stainless steel diffusion cells. After application to the original upper side of the leaf and evaporation of the test liquid, i.e. of the aqueous preparations of the active ingredients without or with the glucamide-containing spray liquids or comparative compositions, the diffusion cells were transferred into thermostatted blocks and charged with aqueous liquid. The water used to make up the aqueous test liquids was local tap water (of known composition). At regular intervals, aliquot samples were taken and the proportion of active ingredient penetrated was determined by HPLC. During the experiment, the temperature in the system (block, diffusion cells, liquids, etc.) and the air humidity above the spray coating on the cuticle were known exactly and were monitored. In the experiment, relative air humidity was constant throughout at 56% relative air humidity (air over supersaturated calcium nitrate) at a constant 25° C. The analytical determination by means of HPLC (1290 Infinity, Agilent) was effected after with a Kinetex column 30×2, 1 mm, 2.6μ C18 100A (Phenomenex), taking a 20 μL aliquot as injection volume at the specified times. In each case, the geometric mean values of the penetration for intact membranes at the mean measurement times are given. According to the variant (active ingredient×test additive/formulation), 7-8 repetitions were set up. The coefficient of variation was below 35%, which is a typical biological variability for penetration for numerous plants (Baur, 1997).

TABLE 1
Penetration of mesotrione (active ingredient concentration 0.3 g/L in
spray liquid) in the presence of 0.75 g/L ammonium sulfate and of the
glucamides Synergen GA (N-methyl-N-octanoyl/decanoylglucamine),
N-methyl-N-dec-9-enoylglucamine and N-methyl-N-nonanoylglucamine
(all with 50% active substance)

| | Spray liquor | Mean penetration in % after | |
|---|---|---|---|
| Test product | concentration | time (n = 4-8) 0.3 g/L ai | |
| (conc.) | [%] | 12-14 h | 1 day |
| Synergen GA | 0.1 | 3.4 | 5.8 |
| Synergen GA | 0.25 | 4.4 | 7.1 |
| Synergen GA | 0.5 | 3.9 | 8.2 |
| N-Methyl-N-dec-9- | 0.1 | 3.5 | 7.3 |
| enoylglucamine | | | |
| N-Methyl-N-dec-9- | 0.25 | 6.1 | 15.0 |
| enoylglucamine | | | |
| N-Methyl-N-dec-9- | 0.5 | 6.0 | 12.0 |
| enoylglucamine | | | |
| N-Methyl-N- | 0.1 | 3.1 | 6.7 |
| nonanoylglucamine | | | |
| N-Methyl-N- | 0.25 | 2.3 | 4.4 |
| nonanoylglucamine | | | |
| N-Methyl-N- | 0.5 | 6.7 | 11.5 |
| nonanoylglucamine | | | |

* 25° C./56% rel. air humidity

The table shows that N-methyl-N-dec-9-enoylglucamine led to higher promotion of penetration of mesotrione compared to the linear mixture of C8 and C10 glucamide. N-Methyl-N-nonanoylglucamine, which was likewise tested as well, likewise falls short.

### Example 3 Dynamic Surface Tension (Interfacial Activity)

In the case of plants that are difficult to wet, such as the cereal plants wheat, barley, triticale, rye and oats, in the case of further large-scale crops corn, rice, soya and oilseed rape, and also in the case of almost all weed grasses and numerous dicotyledonous weeds that are difficult to control, such as Chenopodium album or Euphorbium heterophyllum, the promotion of the adsorption of the spray liquid on the green parts of the plant is of crucial significance. This wetting agent effect was therefore also determined for the glucamides.

For a given application technique or parameters (nozzle, pressure, water application rate, distance from the plant surface), the value for the dynamic surface tension in [mN/m] correlates well with the adhesion on plants that are difficult to wet such as barley (or cereals in general) and weed grasses. A value of 50 mN/m (at 20-21° C.) with respect to water (72.8 mN/m) results in an improvement in the adhesion from “zero adhesion” to about 50% (Baur P, Pontzen R 2007. Basic features of plant surface wettability and deposit formation and the impact of adjuvants. In: R E Gaskin ed. Proceeding of the 8th International Symposium on Adjuvants for Agrochemicals. Publisher: International Society for Agrochemical Adjuvants (ISAA), Columbus, Ohio, USA). A value below 60 mN/m at 200 ms gives visibly better adsorption of aqueous spray liquids; in the case of standard flat jet nozzles, optimal wetting is achieved.

The positive wetting and sticking effects do of course also apply to other organisms and synthetic surfaces or technical applications, for instance for attainment of thin coatings on or the cleaning of surfaces.

The values for dynamic surface tension (Krüss PocketDyne BP2100, T 24.6° C.) are shown below at for the linear C810 glucamide and the decene glucamide and a pelargonamide.

TABLE
Dynamic interfacial tension of glucamides
(temperature 24.6° C.)

| | Conc. | Surface tension [mN/m] | | | |
|---|---|---|---|---|---|
| Substance | [g/L] | 20 ms | 50 ms | 100 ms | 200 ms |
| Synergen GA | 0.6 | 70.8 | 69.6 | 68.5 | 66.5 |
| Synergen GA | 1.25 | 66.2 | 62.5 | 60.3 | 58.1 |
| Synergen GA | 2.5 | 58.2 | 53.4 | 51.4 | 48.7 |
| Synergen GA | 5 | 45.2 | 41.5 | 39.8 | 38.0 |
| N-Methyl-N-dec-9- | 0.6 | 70.2 | 68.9 | 67.6 | 67.4 |
| enoylglucamine | | | | | |
| N-Methyl-N-dec-9- | 1.25 | 66.4 | 63.7 | 61.8 | 61.0 |
| enoylglucamine | | | | | |
| N-Methyl-N-dec-9- | 2.5 | 59.7 | 56.6 | 55.5 | 54.1 |
| enoylglucamine | | | | | |
| N-Methyl-N-dec-9- | 5 | 50.5 | 48.3 | 47.7 | 46.6 |
| enoylglucamine | | | | | |
| N-Methyl-N- | 0.6 | 70.2 | 68.8 | 67.7 | 67.1 |
| nonanoylglucamine | | | | | |
| N-Methyl-N- | 1.25 | 66.0 | 63.7 | 62.6 | 61.1 |
| nonanoylglucamine | | | | | |
| N-Methyl-N- | 2.5 | 59.1 | 56.3 | 55.3 | 53.8 |
| nonanoylglucamine | | | | | |
| N-Methyl-N- | 5 | 47.7 | 45.6 | 45.2 | 44.3 |
| nonanoylglucamine | | | | | |

The results for dynamic surface tension show that N-methyl-N-dec-9-enoylglucamine is a wetting agent of comparable suitability to Synergen GA (C810). The same applies to the pelargonic acid glucamide.

Example 3 also shows that the surprising result (example 2) of better promotion of penetration of mesotrione by the decene glucamide compared to Synergen GA (C810) does not correlate with the wetting properties.

### Example 4

The plant compatibility of N-methyl-N-dec-9-enoylglucamine and pelargonic acid at the abovementioned concentrations of 0.6, 1.25, 2.5 and 5 g/L is just as good as that of Synergen GA and N-methyl-N-nonanoylglucamine. After application to poinsettia plants of the Princettia variety, there were no necroses or other symptoms in any case, whereas an ethoxylated lauryl alcohol at 1 g/L that was also tested caused distinct necroses within one day.

1. A sugar surfactant of the formula (I) 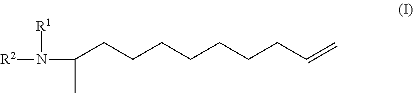
in which
R¹ is H or an alkyl group having 1 to 3 carbon atoms and
R² is a polyhydroxyhydrocarbyl group comprising a linear hydrocarbyl chain, where at least three hydroxyl groups are bonded directly to the chain.

2. The sugar surfactant as claimed in claim 1, wherein
R¹ is an alkyl group having 1 to 3 carbon atoms,
R² is —CH₂—(CHOH)ₙ—CH₂OH, —CH₂—(CH₂OH)(CHOH)ₙ₋₁—CH₂OH, or —CH₂—(CHOH)₂(CHOR³)(CHOH)—CH₂OH,
n is 3, 4 or 5 and
R³ is H or a cyclic mono- or polysaccharide.

3. The sugar surfactant as claimed in claim 1, wherein
R¹ is methyl and
R² is —CH₂—(CHOH)₄—CH₂OH.

4. An aqueous adjuvant composition comprising
A1) at least one sugar surfactant of the formula (I) as claimed in claim 1,
A2) water,
A3) optionally at least one cosolvent,
A4) optionally at least one ammonium salt and
A5) optionally at least one softener.

5. The adjuvant composition as claimed in claim 4, wherein in formula (I)
R¹ is a methyl group and
R² is CH₂—C(CHOH)₄—CH₂OH.

6. The adjuvant composition as claimed in claim 4, wherein the proportion of the at least one sugar surfactant (I) is 10% to 90% by weight, based on the total weight of the composition.

7. The adjuvant composition as claimed in claim 4, wherein the at least one cosolvent (A3) is selected from the group consisting of propylene glycol, dipropylene glycol, mixtures of propylene glycol and dipropylene glycol, each optionally in a mixture with polypropylene glycol and or polyethylene glycol, each having up to ten repeat units.

8. The adjuvant composition as claimed in claim 4, wherein the at least one ammonium salt (A4), is selected from the group consisting of ammonium sulfate, ammonium nitrate, ammonium nitrate urea, ammonium phosphate, ammonium citrate, ammonium chloride and ammonium thiosulfate.

9. A process for enhancement of the biological activity of at least one active agrochemical ingredient, comprising the step of contacting a plant with a composition comprising at least one sugar surfactant of the formula (I) 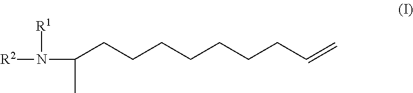
in which
R¹ is H or an alkyl group having 1 to 3 carbon atoms and
R² is a polyhydroxyhydrocarbyl group comprising a linear hydrocarbyl chain,
where at least three hydroxyl groups are bonded directly to the chain,
and the at least one active agrochemical ingredient.

10. A process for production of an aqueous active agrochemical ingredient composition comprising the step of mixing the at least one sugar surfactant of the formula (I) 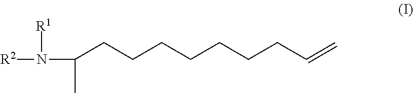
in which
R¹ is H or an alkyl group having 1 to 3 carbon atoms and
R² is a polyhydroxyhydrocarbyl group comprising a linear hydrocarbyl chain,
where at least three hydroxyl groups are bonded directly to the chain,
with the at least one active agrochemical ingredient.

11. An active ingredient composition comprising
W1) at least one sugar surfactant of the formula (I) as claimed in claim 1,
(W2) water,
(W3) at least one soluble active ingredient,
(W4) optionally at least one cosolvent,
(W5) optionally at least one ammonium salts,
(W6) optionally at least one softener,
(W7) optionally at least one auxiliaries, and
(W8) optionally at least one water-insoluble active ingredient.

12. The active ingredient composition as claimed in claim 11, wherein the at least one water-soluble active ingredient is an active agrochemical ingredient.

13. The active agrochemical ingredient composition as claimed in claim 12, wherein the at least one water-soluble active agrochemical ingredient of component (W3) is selected from the group consisting of plant nutrients, plant fortifiers, plant growth regulators and herbicides.

14. The active agrochemical ingredient composition as claimed in claim 12, wherein the at least one water-soluble active agrochemical ingredient of component (W3) is selected from water-soluble salts of 2,4-D, bentazon, dicamba, fomesafen, glyphosate, glufosinate, MCPA, mesotrione, paraquat and sulcotrione.

15. The active agrochemical ingredient composition as claimed in claim 12, further comprising at least one water-insoluble active ingredient.

16. The active agrochemical ingredient composition as claimed in claim 12, wherein the total amount of the active agrochemical ingredients of component (W3) in the composition is greater than 100 g/L, based on the acid equivalent thereof.

17. The active agrochemical ingredient composition as claimed in claim 12, wherein the total amount of the sugar surfactants of the formula (I) in the composition is from 20 to 250 g/L.

18. The active agrochemical ingredient composition as claimed in claim 12, which comprises an ammonium salt (W5).

19. The active agrochemical ingredient composition as claimed in claim 12, which comprises, as well as component (W1), at least one further adjuvants.

20. The active agrochemical ingredient composition as claimed in claim 12, which takes the form of a concentrate formulation which is diluted prior to use and contains 5% to 80% by weight of the at least one water-soluble active agrochemical ingredient of component (W3) and 1% to 25% by weight of the at least one sugar surfactant of component (W1).

21. The active agrochemical ingredient composition as claimed in claim 12, which takes the form of a spray liquor and contains 0.001% to 10% by weight of the at least one water-soluble pesticide of component (W3) and 0.01% to 1% by weight of the at least one sugar surfactant of component (W1).

22. (canceled)

23. A method of protecting plants from harmful organisms, comprising the step of contacting the plant, the harmful organisms or their habitat with an active agrochemical ingredient composition, wherein the active agrochemical ingredient comprises a pesticide, and at least one sugar surfactant of the formula (I) 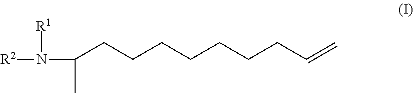
in which
R¹ is H or an alkyl group having 1 to 3 carbon atoms and
R² is a polyhydroxyhydrocarbyl group comprising a linear hydrocarbyl chain,
where at least three hydroxyl groups are bonded directly to the chain.

24. The method as claimed in claim 23 for control and/or for abatement of unwanted plant growth, fungal disorders or insect infestation in plants.

25. The adjuvant composition as claimed in claim 4, wherein the at least one softener is selected from the group consisting of alkali metal phosphates, alkali metal citrates, alkali metal sulfates, glycerol sulfates, phosphonates, aminopolycarboxylic acids, aminopolyphosphonic acids, gluconates, monocarbamide dihydrogensulfate, polymeric polycarboxylates and polymeric polyphosphonates.

26. The active agrochemical ingredient composition as claimed in claim 12, which comprises at least one softener, selected from the group consisting of alkali metal phosphates, alkali metal citrates, alkali metal sulfates, glycerol sulfates, phosphonates, aminopolycarboxylic acids, aminopolyphosphonic acids, gluconates, monocarbamide dihydrogensulfate, polymeric polycarboxylates and polymeric polyphosphonates.
